(19) United States
(12) Patent Application Publication
(10) Pub. No.: US 2016/0144164 A1
(43) Pub. Date: May 26, 2016

(54) IMPLANTABLE MEDICAL LEAD WITH PROTECTIVE TUBING ELEMENT

(71) Applicant: Medtronic Bakken Research Center B.V., Maastricht (NL)

(72) Inventor: Karo Sedighiani, Eindhoven (NL)

(21) Appl. No.: 14/944,892

(22) Filed: Nov. 18, 2015

Related U.S. Application Data
(60) Provisional application No. 62/084,426, filed on Nov. 25, 2014.

(51) Int. Cl. A61N 1/05; A61N 1/36

(52) U.S. Cl. CPC A61N 1/0534; A61N 1/36067; A61N 1/36096

(57) ABSTRACT

A protective tubing element for a medical lead includes an outer flexible tube, and a structurally flexible reinforcement at least partially covered by the at least one outer flexible tube, the outer flexible tube and the structurally flexible reinforcement are arranged such that the structurally flexible reinforcement and the outer flexible tube allow movements relative to each other.

This application claims the benefit of U.S. Provisional Application No. 62/084,426, filed Nov. 25, 2014, the entire content of which is incorporated herein by reference.

### TECHNICAL FIELD

This disclosure relates to medical leads.

### BACKGROUND

Implantable neurostimulation devices may treat acute or chronic neurological conditions. Deep brain stimulation (DBS), the mild electrical stimulation of sub-cortical structures, belongs to this category of implantable devices, and has been shown to be therapeutically effective for Parkinson's disease, Dystonia, and Tremor. In addition, DBS may be used to treat psychiatric disorders (e.g., obsessive compulsive disorder, depression). DBS systems generally include one or more leads connected to an implantable pulse generator.

### SUMMARY

In one example, this disclosure is directed to a medical lead including an outer flexible tube, and a structurally flexible reinforcement at least partially covered by the outer flexible tube, wherein the structurally flexible reinforcement and the outer flexible tube are arranged such that the structurally flexible reinforcement and the outer flexible tube allow movement relative to each other, and wherein the protective tubing element is configured to cover a proximal portion of the medical lead.

In a further example, this disclosure is directed to a system comprising a medical lead, at least one protective tubing element of the proceeding paragraph, and electronics for driving the lead electrically coupled to electrodes of the medical lead.

In another example, this disclosure is directed to an system including a medical lead comprising a lead body having a proximal end and a distal end; a plurality of electrodes at the distal end of the lead body; a plurality of conductors extending from the electrodes to the proximal end of the lead body; a controller configured to deliver electrical stimulation current via the plurality of electrodes of the medical lead; and a protective tubing element comprising: an outer flexible tube; and a structurally flexible reinforcement at least partially covered by the outer flexible tube, wherein the structurally flexible reinforcement and the outer flexible tube are arranged such that the structurally flexible reinforcement and the outer flexible tube allow movements relative to each other, and wherein the protective tubing element is configured to cover a proximal portion of the medical lead.

In another example, this disclosure is directed to a system including a medical lead; and a protective tubing element comprising: an outer flexible tube; an inner flexible tube; and a structurally flexible reinforcement sandwiched between the outer flexible tube and the inner flexible tube, wherein the structurally flexible reinforcement and the outer flexible tube are arranged such that the structurally flexible reinforcement and the outer flexible tube allow movements relative to each other, and wherein the protective tubing element is configured to cover a proximal portion of the medical lead.

The details of one or more examples of this disclosure may be set forth in the accompanying drawings and the description below. Other features, objects, and advantages of this disclosure may be apparent from the description and drawings, and from the claims.

### BRIEF DESCRIPTION OF DRAWINGS

FIG. 1 is a conceptual illustration of a neurostimulation system for DBS.

FIG. 2 is a conceptual illustration of a lead system.

FIG. 3 a functional block diagram illustrating components of an example medical device system including an implantable pulse generator and a separate active lead can (ALC) with a switch matrix to direct signals from the implantable pulse generator to different electrodes.

FIG. 4 is a conceptual illustration of an implanted lead with a protective tubing element.

FIG. 5 is a conceptual illustration drawing of a lead of a neurostimulation system for DBS and its components.

FIG. 6 is a perspective view of a protective tubing element.

FIG. 7 is a cross-sectional side view of the protective tubing element in the moment after an impact.

FIG. 8 is a cross-sectional side view of the protective tubing element in the moment of bending.

FIG. 9 is a cross-sectional side view of the protective tubing element in the moment of bending.

### DETAILED DESCRIPTION

Implantable neurostimulation devices may treat acute or chronic neurological conditions. DBS, the mild electrical stimulation of sub-cortical structures, belongs to this category of implantable devices, and has been shown to be therapeutically effective for Parkinson's disease, Dystonia, and Tremor. In addition, DBS has been proposed for use to treat psychiatric disorders (e.g., obsessive compulsive disorder, depression). The technology used in the field of DBS can also be used for spinal cord stimulation (SCS), vagus nerve stimulation (VNS), pelvic floor stimulation, gastric stimulation, peripheral nerve stimulation, or other stimulation or sensing utilizing medical leads, including, but not limited to, cardiac stimulation and sensing.

The present disclosure relates to a protective tubing element for a lead for neurostimulation and/or neurorecording, the protective tubing element including an outer flexible tube and, a structurally flexible reinforcement at least partially covered by the outer flexible tube. The outer flexible tube and the structurally flexible reinforcement tube are arranged such that the structurally flexible reinforcement and the outer flexible tube allow movements relative to each other. In some examples, the outer flexible tube and the structurally flexible reinforcement are not connected. In some examples, there may be a single connection between the outer flexible tube and the structurally flexible reinforcement. In other examples, the protective tubing element may include spaced connections between the outer flexible tube and the structurally flexible reinforcement. The present disclosure further relates to a protective tubing element for a lead of a system for neurostimulation and/or neurorecording and a system for neurostimulation and/or neurorecording.

The techniques of this disclosure may be implemented in combination with systems including smaller electrodes, such as electrodes manufactured using thin film manufacturing. Examples of manufacturing techniques for a lead made from a thin film based on thin film technology are disclosed in published international application no. WO 2010/055453 A1, entitled “Spiraled wires in a deep-brain stimulator probe,” to Harberts et al., the entire contents of which is incorporated by reference herein. The thin film carries multiple electrodes to cover the distal tip with an array of electrodes, and is assembled into a lead. In some examples, the array of electrodes may be a complex array comprising electrode segments arranged at different axial positions along the length of the length and at different angular positions around the circumference of the lead. Such leads may enhance the precision to address the appropriate target in the brain and relax the specification of positioning. Meanwhile, undesired side effects due to undesired stimulation of neighboring areas can be minimized. The thin film leads may be fixed on a core material to form a lead. Examples of such manufacturing techniques for a lead based on thin film manufacturing are disclosed in U.S. Pat. No. 7,941,202, entitled “Modular multichannel microelectrode array and methods of making same,” to Hetke et al., the entire content of which is incorporated by reference herein.

In some examples, a DBS lead includes four 1.5 mm-wide cylindrical (i.e., “ring”) electrodes at the distal end spaced apart from one another, e.g., axially, by 0.5 mm or 1.5 mm. In this manner, the cylindrical electrodes may be provided at a plurality of axial positions along the length of the lead. In one example, the diameter of the lead is approximately 1 to 1.5 mm, and more preferably approximately 1.27 mm. In some examples, the diameter of the lead may be up to approximately 4 mm. The lead may encompass interconnect wires connecting the electrodes at the distal end of the lead to an active lead can at the proximal end of the lead. The metal used for the electrodes and interconnect wires connecting the electrodes to the active lead can are metal, such as an alloy of platinum and iridium. In other examples, the cylindrical electrodes may be segmented to provide individual electrode segments at different angular positions around the circumference of the lead. The interconnect wires may be insulated individually by a fluoropolymer coating. A urethane tubing of a few tens of microns thick over the fluoropolymer coating may also protect the interconnect wires. With such an electrode design, the current distribution emanates uniformly around the circumference of the electrode, which leads to stimulation of all areas surrounding the electrode.

The lack of fine spatial control over current and electric field distributions implies that stimulation easily spreads into adjacent structures inducing adverse side-effects in as much as 30% of the patients. Systems with high density electrode arrays may overcome this problem by providing the ability to steer the stimulation field to the appropriate target (hence the term steering brain stimulation).

The clinical benefit of DBS may be largely dependent on the spatial distribution of the stimulation field in relation to brain anatomy. To improve therapeutic benefits while limiting unwanted side-effects, precise control over the stimulation field is important.

During stimulation with existing DBS leads there is an option to use monopolar, bipolar, or even multipolar stimulation. Neurostimulator devices with steering brain stimulation capabilities can have a large number (n) of electrode contacts (n>10) that can be connected to electrical circuits such as current or voltage sources and/or (system) ground. Stimulation may be considered monopolar when the distance between the anode and cathode may be several times larger than the distance of the cathode to the stimulation target. During monopolar stimulation in homogeneous tissue, the electric field may be distributed roughly spherically, similar to the field from a point source. When the anode is located close to the cathode, i.e., in a bipolar configuration, the distribution of the field becomes more directed in the anode-cathode direction. As a result, the field gets stronger and neurons may be more likely to be activated in this area due to a higher field gradient.

Polarization (de- and/or hyperpolarization) of neural tissue may play a prominent role both for suppression of clinical symptoms, and for induction of stimulation-induced side-effects. In order to activate a neuron it has to be depolarized. Neurons are generally depolarized more easily close to the cathode than by the anode (about 3-7 times more depending on type of neuron, etc.).

A DBS lead is implanted in to a burr hole through the skull and the cranium into the brain. The lead itself may have a diameter at or below approximately 4 mm and thus protection from mechanical stress may be important to avoid mechanical failure as the proximal part of the lead in the region of the cranium and outside of the cranium and the skull may be subject to mechanical forces and stress. In some examples, the diameter of the lead is between approximately 1 mm and 1.5 mm. In accordance with an example of this disclosure, a protective tubing element for a lead for neurostimulation and/or neurorecording may limit such mechanical forces and stress. In particular, the proximal part of the lead may be protected in the region of the cranium and outside of the cranium and the skull from mechanical forces and stress by the protective tubing element. Accordingly, a protective tubing element for a lead for neurostimulation and/or neurorecording may include an outer flexible tube and a structurally flexible reinforcement at least partially covered by the at least one outer flexible tube. The outer flexible tube and the structurally flexible reinforcement may move freely within respect to one another.

A lead inside the protective tubing element may be protected from mechanical forces and stress by the protective tubing element dissipating or receiving the mechanical stressors which would otherwise be applied to the lead. For example, the proximal part of the lead may be protected in the region of the cranium and outside of the cranium and the skull from mechanical forces and stress by the protective tubing element.

The lead may be a lead based on thin film technology with thin film tracks and a plurality (n) of electrodes (e.g., number of electrodes n>10) connected with tracks in the thin film. Such a thin film can be damaged by excessive stress, leading to implant failure. In particular, the region of the lead running subcutaneously from the proximal side of the burr hole in the skull and cranium is potentially exposed to shocks, impact loads or repetitive strains, bendings, or other stressors. For instance, the lead may undergo an approximately ninety degree bend as it exits the burr hole and traverses along the skull.

The protective tubing element may be configured to resist impact loads without shearing collapse and without high plastic deformation or skin penetration. The protective tubing element may include an outer protective layer and a structurally flexible reinforcement member. The structurally flexible reinforcement member is configured to balance additional impact resistance while maintaining structural flexibility. In some examples, this is achieved by using a material with a relatively high impact resistance and configuring the material in a coil or braided mesh, wherein the structure of the reinforcement provides flexibility not otherwise present in a solid tube made of the material. Further, by minimizing the connections between an outer protective layer and a structural flexible reinforcement member, the protective tubing element may bend without portions of the outer protective layer being pinched in between portions of the structural flexible reinforcement member. The protective tubing element may be configured to resist impact loads while keeping the flexibility comparable to the lead without the protective tubing element. Furthermore, the total outer diameter of the protective tubing element may be kept compatible with the implantable dimension, e.g., only at a few millimeters and at low wall thicknesses. Additionally, high axial stiffness is provided.

The protective tubing element may be configured to provide an impact resistance of approximately 0.1 up to 6 Joules or more, e.g., the protective tubing element is capable to protect against impact scenarios with an impact energy of approximately 0.1 to 6 Joules. In some examples, the protective tubing element is configured to provide an impact resistance of approximately 0.5 to 2.5 Joules. In some examples, the protective tubing element may be configured to meet standards for impact resistance for a variety of implants. Although discussed primarily with respect to a lead for DBS, the lead or electrode system protected by the protective tubing element may be used in conjunction with other implants, including, for example, cochlear implants, heart implants, spinal cord stimulators, pelvic floor stimulators, gastric stimulators, and visual implants. The protective tubing may be configured to meet any implant standard associated with the type of implant being protected. For example, the protective tubing element may be configured to meet an applicable cochlear implant impact standard of approximately 2.5 Joules.

The structurally flexible reinforcement of the protective tubing element may be at least partially a coil. For example, the structurally flexible reinforcement elements may be a helically wound coil. Generally speaking, the cross-section of the coil can be chosen on demand and the selection of the cross-section is not limited to a particular shape. For example, the cross-section may be elliptical, circular, rectangular, rhombic, or trapezoidal. In some examples, the structurally flexible reinforcement may include a flexible tube shaped structure, like a laser-cut tube, micro-processed tube, or 3D printed tube.

In examples of the protective tubing element including both an inner flexible tube and an outer flexible tube, the structurally flexible reinforcement may be freely sandwiched between the inner flexible tube and the outer flexible tube. This increases flexibility and allows movement of the elements of the protective tubing element. The protective tubing element is designed to provide an overall structure that may provide flexibility on the one hand and stability and sufficient protection on the other hand. In examples including only an outer flexible tube and a structurally flexible reinforcement, the outer flexible tube and the structurally flexible reinforcement may move freely with respect to one another.

It is of course possible to partially attach the structurally flexible reinforcement to the inner flexible tube and/or outer flexible tube, e.g., by gluing. However, the structurally flexible reinforcement and the inner and outer tube may still be able to perform relative movements with respect to each other.

The structurally flexible reinforcement may provide gaps or interspaces. In some examples, the inclusion of gaps or interspaces may allow the structurally flexible reinforcement to be made of a material with a relatively higher impact resistance, while still maintaining structural flexibility. Such gaps or interspaces may be configured to allow for greater flexibility to a normally relatively inflexible material. The gaps or interspaces may also be configured to be sufficiently large to provide enough space for, e.g., either the outer flexible tubing element or the inner flexible tubing element to deform within without pinching when either the outer tubing element or the inner tubing element is pressed and partially deformed in the direction of the structurally flexible reinforcement upon impact or mechanical force or bending. If the gaps or the interspaces may be sufficiently large enough, the deformation of either the outer flexible tube or the inner flexible tube does not affect or does not substantially affect the structural integrity of the other flexible tube. The pitch of the structurally flexible reinforcement coil may be, for example, in the range of the width or between a range of 0.5 to 2.5 times the width of a wire forming the coil. For example, a pitch of around 1.2 times of the width offers a good structural flexibility.

The inner flexible tube and/or the outer flexible tube of the protective tubing element may be at least partially made of a flexible polymer. In particular, the flexible polymer may be a biocompatible and/or hemocompatible material. The flexibility of the flexible polymer of the inner flexible tube and/or the outer flexible tube of the protective tubing element allows the lead encapsulated by the protective tubing element to be bent or otherwise manipulated during implantation or use.

The flexible polymer used to form the inner flexible tube and/or the outer flexible tube is or at least partially comprises an elastomer or a thermoplastic urethane, a polyurethane or a polycarbonate-urethane, for example. These polymers potentially provide an advantageous flexibility, good biocompatibility and do not cause tissue reactions at the implantation site.

The axial strain limit of the flexible polymer used to form the inner flexible tube and/or the outer flexible tube may be higher than 75%. Polymers with such an axial strain limit may be especially adapted to cope with the possible bending and forces. Furthermore, such polymers show better results in impact scenarios than polymers with lower axial strain limits.

The structurally flexible reinforcement may be at least partially made of metal or a metal alloy or a polymer or a reinforced polymer. The structurally flexible reinforcement may be made of any relatively stiff material. For example, it can be metal or metal alloy, high stiffness polymers like PEEK (e.g. thermoplastic polymer) or epoxy (e.g. thermosets polymer), composite material, fiber-reinforced materials like CFK-materials, and the like. In some examples, the structurally flexible reinforcement may be made of a biocompatible material. The selection of material type may be made by balancing how much flexibility is desired against the impact resistance desired. In some examples, a metal used to form the structurally flexible reinforcement may be titanium. Alternatively, the structurally flexible reinforcement may be formed from a metal alloy, which may be a nickel alloy like MP35N. Theses metals/metal alloys may be very suitable to form the structurally flexible reinforcement, e.g., in the form of a coil or the like. In some examples, the strain limit and yield stress of the structurally flexible reinforcement may be controlled by aging and heat treatment of the metal alloy. In some examples, the MP35N used in the structurally flexible reinforcement has an approximately 1200 megapascal yield stress and approximately 20% failure strain.

The outer diameter of protective tubing element may be, for example, in the field of DBS between approximately 2.0 mm to approximately 2.5 mm, such as between approximately 2.3 to approximately 2.4 mm. These dimensions may be acceptable for implantation in order to perform a minimally invasive intervention for the lead implantation. In other application fields like spinal cord stimulation (SCS) or vagus nerve stimulation (VNS), different, especially larger, diameters for the outer diameter of the protective tubing element may be used. For examples, for leads with a diameter of approximately 4 mm, the outer diameter of the protective tubing element may be approximately 5 mm.

The wall thickness of the inner flexible tube and/or the at least one outer flexible tube may be, for example, in the field of DBS, between approximately 0.01 mm to approximately 1 mm, such as between approximately 0.05 mm to approximately 0.15 mm. The wall thickness of the at least one structurally flexible reinforcement is between approximately 0.05 mm to approximately 1.25 mm, such as between approximately 0.1 mm to approximately 0.5 mm. The total thickness of the protective tubing element is between approximately 0.07 mm and approximately 3 mm, for example between approximately 0.1 mm and approximately 0.6 mm with an outer diameter of the protective tubing element between approximately 2.0 to approximately 2.4 mm for use in connection with DBS stimulation.

In other application fields like spinal cord stimulation (SCS) or vagus nerve stimulation (VNS), different, thicker protective tubing elements may be used, resulting in a larger overall diameter. These ranges of thickness of the tubes and the structurally flexible reinforcement may be selected to provide adequate impact resistance while maintaining small dimensions needed for minimally invasive surgery.

In some examples, the structurally flexible reinforcement may be a helical coil. The cross-section of the structurally flexible reinforcement may be substantially rectangular or rectangular. For example, a helical coil with a rectangular cross-section may be created through the use of a laser cut to a tube. For example, the tube itself may have a circular cross-section when viewed from the end of the tube, but the cross-section of the structurally flexible reinforcement is substantially rectangular. This increases the stability of the structurally flexible reinforcement, especially when the structurally flexible reinforcement is a coil. Alternatively, the cross-section of the least one structurally flexible reinforcement may be elliptical, circular, rectangular, rhombic, or trapezoidal. In other examples, the structurally flexible reinforcement may be a braided mesh. The structurally flexible reinforcement provides the protective tubing element with higher impact resistance against compression than the outer tube alone. The structurally flexible reinforcement provides stiffness with minimal compromise to the overall flexibility of the protective tubing element.

The lead protected by the protective tubing element may be for neurostimulation and/or neurorecording and may be part of a neurostimulation and/or neurorecording system. In some examples, the neurostimulation system may be for DBS.

In some examples, the tissue surrounding the neurostimulation system components, including the protective tubing element and lead, may be brain tissue (in connection with DBS) or for example any other neural tissue, like the spinal cord. Other applications can be possible like for cochlear implants, heart implants, visual implants, for example.

Moreover, the plurality of electrodes associated with the neurostimulation system allow steering of the stimulation field such that the stimulation field may be adapted and conform with a target area. In particular, stimulation steering is enabled where the electric field is shaped such that certain directions receive much less stimulation current than other adjacent tissue regions.

In some examples, the lead may include at least 10 electrodes, more preferably at least 20 electrodes, and more preferably approximately 30 to 45 electrodes, and still more preferably approximately 40 electrodes, although greater numbers of electrodes are possible. This number of electrodes allows the creation of stimulation field which conforms to the target region and which may be formed three-dimensionally and adapted to the target region. Only those regions that need to be stimulated may be covered by the stimulation field provided by the plurality of electrodes.

The electrodes may form a complex electrode array. This is helpful to create a stimulation field that is adapted to and conforms with the target region. A complex electrode array generally refers to an arrangement of electrodes at multiple non-planar or non-coaxial positions, in contrast to simple electrode array geometries in which the electrodes share a common plane or common axis.

An example of a simple electrode array geometry is an array of ring electrodes distributed at different axial positions along the length of a lead. An example of a complex electrode array geometry, in accordance with this disclosure, is an array of electrodes positioned at different axial positions along the length of the lead, as well as at different angular positions about the circumference of e.g. lead of the neurostimulation system. Hence, in some examples, a complex electrode geometry may permit directional steering of stimulation fields, e.g., in axial and radial directions relative to the longitudinal axis of the lead. In some examples, the electrodes may include one or more full ring electrodes in combination with one or more segmented electrodes. An example would be a “1-3-3-1” lead having a distal ring electrode, two rows each having three segmented electrodes, and a more proximal ring electrode. Such a lead is described in U.S. Pat. No. 7,668,601 assigned to the assignee of the current application. In still other examples, the complex electrode array may comprise electrodes formed using thin film techniques and the array may comprise any number of electrodes, such as forty or more electrodes.

Furthermore, the present disclosure relates to a lead for a system for neurostimulation and/or neurorecording, comprising at least one protective tubing element as specified. The protective tubing element may cover a proximal part of the lead, especially wherein the distal part of the lead is not covered by the protective tubing element. The most distal part of the protective tubing element may have a decreasing diameter. This is especially beneficial, when implanting the lead together with the protective tubing element into tissue.

Moreover, the present disclosure relates to a system for neurostimulation and/or neurorecording, comprising at least one protective tubing element as specified.

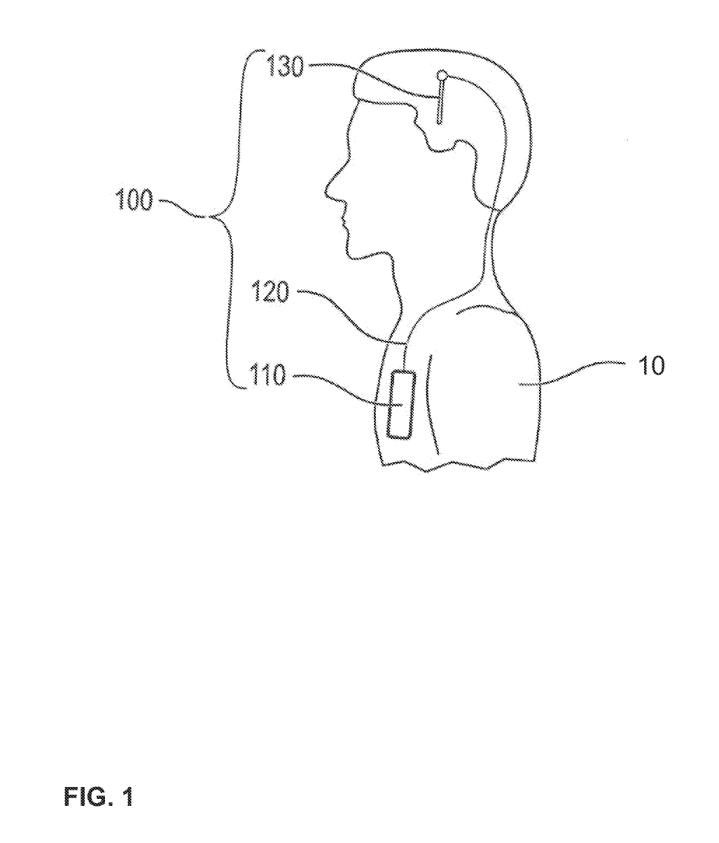

FIG. 1 is a conceptual illustration of a neurostimulation system for DBS consistent with this disclosure. Although neurostimulation system 100 is discussed with respect to providing DBS, other applications of a neurostimulation system are possible.

The neurostimulation and/or neurorecording system 100 includes at least a controller 110 that may be surgically implanted in the chest region of a patient 10, such as below the clavicle or in the abdominal region of a patient 10. The controller 110 can be adapted to supply the necessary stimulation. Controller 110 may be, for example, an implantable pulse generator (IPG) or an implantable medical device (IMD). In some examples the stimulation may include voltage pulses. In other examples, the stimulation may include current pulses. In still other examples, a continuous waveform may be provided (e.g., a sine wave). The example DBS system 100 may further include an extension wire 120 (also referred to herein as a lead extension) connected to the controller 110 and running subcutaneously to the skull, preferably along the neck, where it terminates in a connector. The connector couples to a counterpart connector at the proximal end of a DBS lead arrange 130. The DBS lead arrangement 130 may be implanted in the brain tissue, e.g. through a burr-hole in the skull. DBS lead arrangement 130 is at least partial covered by a protective tubing element (not shown). In some examples, system 100 may also include an external programmer (not shown).

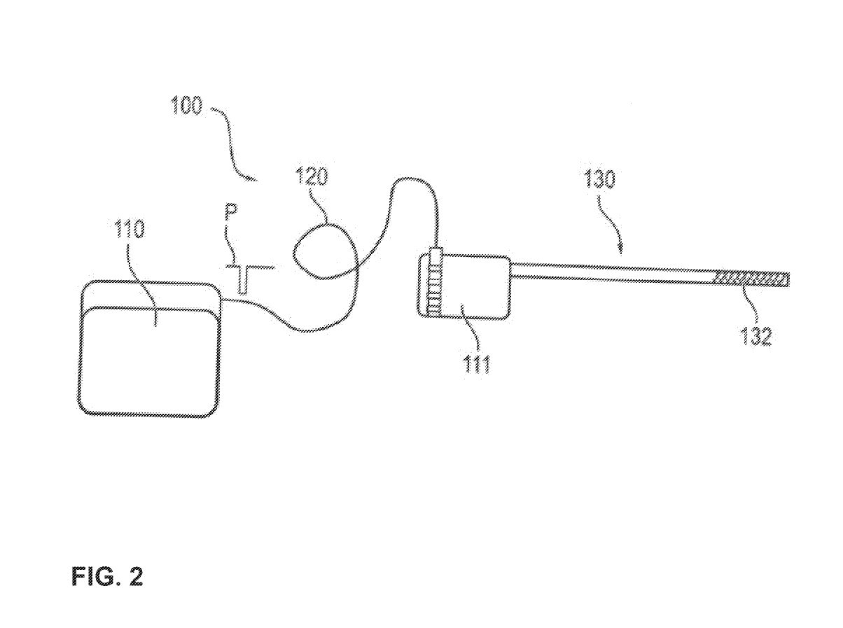

FIG. 2 illustrates an example of a system 100 for brain applications, here for neurostimulation and/or neurorecording as a DBS system 100 as shown in FIG. 1. The system 100 includes at least one lead 130 for brain applications with stimulation and/or recording electrodes 132, wherein e.g. a plurality of electrodes 132 can be provided on outer body surface at the distal end of the lead 130. In some examples, lead 130 may include 40 or more electrodes. Lead 130 comprises an active lead can (ALC) 111. Conductors in ALC 111 may extend (e.g., as by feedthrough connections) to conductors carried by the proximal portion of lead 130. The conductors may extend to a connector at the proximal end, which coupled to a counterpart connector at the distal end of extension wire 120. By means of respective conductors carried by the extension wire, the conductors of lead 130 may be coupled to electronics in controller 110. In this manner pulses P or other signals supplied by controller 110 can be transmitted to ALC 111 and in turn transmitted to electrodes 132. Alternatively or additionally, signals sensed by electrodes 132 can be provided to controller 110. The controller 110 can be an implantable pulse generator or an external trialing system used to deliver signals to electrodes 132 on a short-term trial basis, for example.

In an alternative embodiment, ALC 111 may include a pulse generator or other electronics to generate a pulse train or another type of signal to provide to electrodes 132 and/or to receive and process signals from electrodes 132. In this manner, a signal generator and signal processor may be provided by controller 110, ALC 111, or both. Lead 130 is at least partially covered by a lead protective tubing element (not shown). The lead protective tubing element is configured to resist impact loads without shearing collapse, without high plastic deformation, and without skin penetration, while maintaining flexibility thereby allowing lead 130 to be implanted as desired.

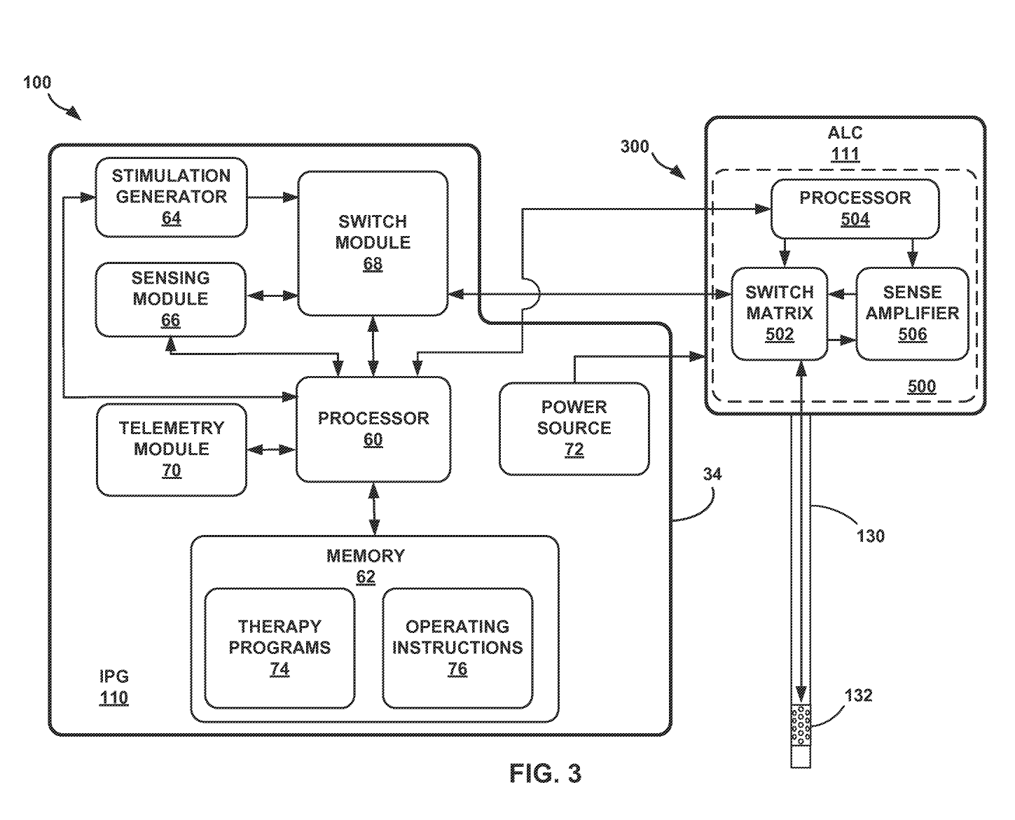

FIG. 3 is functional block diagram illustrating components of an example therapy system 100 including controller 110 and ALC 111. In the example shown in FIG. 3, controller 110 includes processor 60, memory 62, stimulation generator 64, sensing module 66, switch module 68, telemetry module 70, and power source 72. Memory 62, as well as other memories described herein, may include any volatile or non-volatile media, such as a random access memory (RAM), read only memory (ROM), non-volatile RAM (NVRAM), electrically erasable programmable ROM (EEPROM), flash memory, and the like. Memory 62 may store computer-readable instructions that, when executed by processor 60, cause controller 110 to perform various functions described herein.

In the example shown in FIG. 3, memory 62 stores therapy programs 74 and operating instructions 76, for example, in separate memories within memory 62 or separate areas within memory 62. Each stored therapy program 74 defines a particular program of therapy in terms of respective values for electrical stimulation parameters, such as an electrode combination, current or voltage amplitude, and, if stimulation generator 64 generates and delivers stimulation pulses, the therapy programs may define values for a pulse width, and pulse rate of a stimulation signal. The stimulation signals delivered by controller 110 may be of any form, such as stimulation pulses, continuous-wave signals (e.g., sine waves), or the like. Operating instructions 76 guide general operation of controller 110 under control of processor 60, and may include instructions for monitoring brain signals within one or more brain regions via electrodes 132 and delivering electrical stimulation therapy to patient 12.

Stimulation generator 64, under the control of processor 60, generates stimulation signals for delivery to patient 12 via selected combinations of electrodes 132. In some examples, stimulation generator 64 generates and delivers stimulation signals to one or more target regions of brain 28 (FIG. 1), via a select combination of electrodes 132, based on one or more stored therapy programs 74. Processor 60 selects the combination of electrodes 132 with control signals to processor 504 of ALC 111. In turn, processor 504 of ALC 111 selectively activates active switch matrix 504 to direct the stimulation signals received from stimulation generator 64 to the selected electrodes 132. The stimulation parameter values and target tissue sites within brain 28 for stimulation signals or other types of therapy may depend on the patient condition for which therapy system 100 is implemented to manage.

The processors described in this disclosure, including processor 60 and processor 504, may include one or more digital signal processors (DSPs), general-purpose microprocessors, application specific integrated circuits (ASICs), field programmable logic arrays (FPGAs), or other equivalent integrated or discrete logic circuitry, or combinations thereof. The functions attributed to processors described herein may be provided by a hardware device and embodied as software, firmware, hardware, or any combination thereof. Processor 60 is configured to control stimulation generator 64 according to therapy programs 74 stored by memory 62 to apply particular stimulation parameter values specified by one or more therapy programs.

Processor 60 may control switch module 68 to select stimulation generator 64 or sensing module 66. In turn, processor 60 directs processor 504 of electronic module 500 to apply the stimulation signals generated by stimulation generator 64 to selected combinations of electrodes 132, or to sense signals from selected combinations of electrodes 132 via sense amplifier 506 of electronic module 500. In particular, active switch matrix 502 of electronic module 500 may couple stimulation signals to selected conducting tracks within lead 130, which, in turn, deliver the stimulation signals to selected electrodes 132. Hence, although there may be many, for example, 40, electrodes, active switch matrix 502 may select a subset of one, two or more electrodes for delivery of stimulation pulses. Active switch matrix 502 may be a switch array, an array of one or more transistors such as Field-Effect Transistors (FETs) switch matrix, multiplexer, and/or demultiplexer, or any other type of switching module configured to selectively couple stimulation energy to selected electrodes 132 and to selectively sense bioelectrical brain signals with selected electrodes 132. Hence, stimulation generator 64 is coupled to electrodes 132 via switch module 68, conductors between controller 110 and ALC 111, active switch matrix 502, and conducting tracks within lead 130. Additionally, the logic path between stimulation generator and electrodes 132 may include one or more discrete components such as capacitors, resistors, logic gates, transistors, etc. Thus, it will be understood that when reference is made to coupling of stimulation generator 64 or other components of controller 110 to electrodes 132, this refers to the enabling of a logic path between the logic components so that signals may be transferred there between, and is not intended to necessarily require a direct electrical coupling of the components.

In some examples, however, controller 110 does not include switch module 68 and all switching functions may be performed by active switch matrix 502. For example, controller 110 may include multiple sources of stimulation energy (e.g., current sources). Stimulation generator 64 may be a single channel or multi-channel stimulation generator. In particular, stimulation generator 64 may be capable of delivering a single stimulation pulse, multiple stimulation pulses or continuous signal at a given time via a single electrode combination or multiple stimulation pulses at a given time via multiple electrode combinations. In some examples, however, stimulation generator 64 and active switch matrix 502 may be configured to deliver multiple channels on a time-interleaved basis. For example, active switch matrix 502 may serve to time divide the output of stimulation generator 64 across different electrode combinations at different times to deliver multiple programs or channels of stimulation energy to patient 12.

In addition to, or instead of stimulation generator 64 of controller 110, a stimulation generator (not shown) may reside within ALC 111 and may generate the stimulation pulses that are routed to electrodes 132 via active switch matrix 502. In such cases, the stimulation generator within the ALC may receive power from power source 72 and may receive control signals from stimulation generator 64 or other logic of controller 110. The stimulation generator in ALC 111 may be provided in addition to, or instead of, stimulation generator 64 of controller 110. Thus, electronics for lead 130 and electrodes 132 may reside in controller 110, ALC 111, or some combination thereof. As is the case with any stimulation generator 64 of controller 110, any stimulation of ALC 111 may be a single channel or multi-channel stimulation generator as set forth above.

Sensing module 66, under the control of processor 60, is configured to sense bioelectrical brain signals of patient 12 via active switch matrix 502, sense amplifier 506, and a selected subset of electrodes 132 or with one or more electrodes 132 and at least a portion of a conductive outer housing 34 of controller 110, at least a portion of a conductive outer housing of ALC 111, an electrode on outer housing 34 of controller 110, an electrode on an outer housing of ALC 111, or another reference. Processors 60 and 504 may control switch module 68 and active switch matrix 502 to electrically connect sensing module 66 to selected electrodes 132 via active switch matrix 502 and sense amplifier 506 of ALC 111. In this way, sensing module 66 may selectively sense bioelectrical brain signals with different combinations of electrodes 132.

Telemetry module 70 is configured to support wireless communication between controller 110 and an external programmer 14 or another computing device under the control of processor 60. Processor 60 of controller 110 may receive, as updates to programs, values for various stimulation parameters from programmer 14 via telemetry module 70. The updates to the therapy programs may be stored within therapy programs 74 portion of memory 62. Telemetry module 70 in controller 110, as well as telemetry modules in other devices and systems described herein, such as programmer 14, may accomplish communication by RF communication techniques. In addition, telemetry module 70 may communicate with external medical device programmer 14 via proximal inductive interaction of controller 110 with programmer 14. Accordingly, telemetry module 70 may send and receive information to/from external programmer 14 on a continuous basis, at periodic intervals, or upon request from controller 110 or programmer 14.

Power source 72 delivers operating power to various components of controller 110. Power source 72 may include a small rechargeable or non-rechargeable battery and a power generation circuit to produce the operating power. Recharging may be accomplished through proximal inductive interaction between an external charger and an inductive charging coil within controller 110. In some examples, power requirements may be small enough to allow controller 110 to utilize patient motion and implement a kinetic energy-scavenging device to trickle charge a rechargeable battery. In other examples, traditional batteries may be used for a limited period of time.

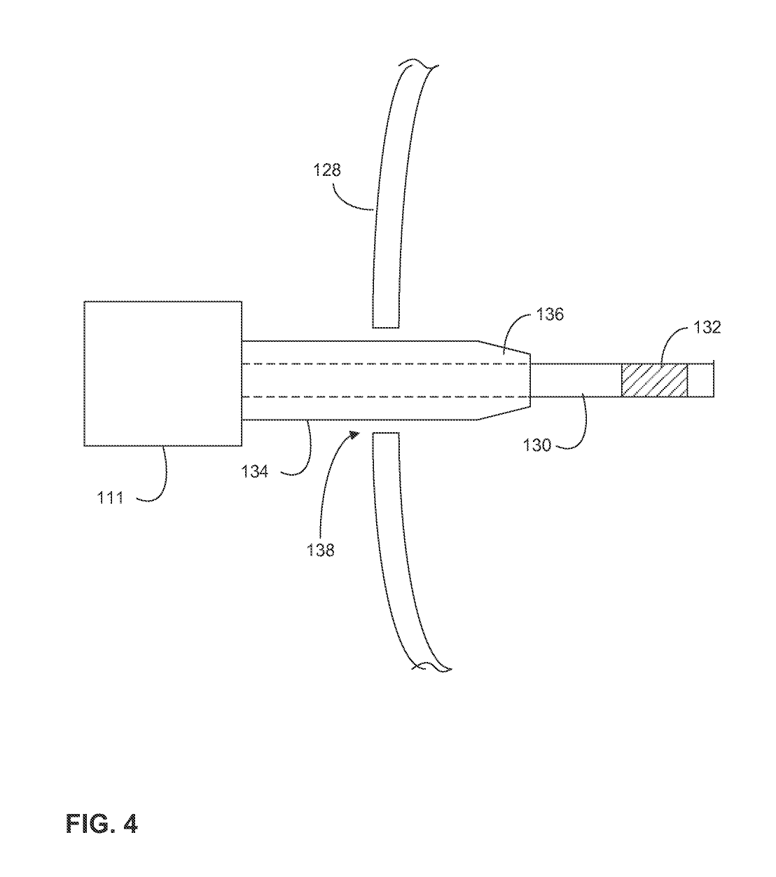

FIG. 4 shows a conceptual illustration of an implanted lead 130 connected to active lead can 111 and at least partially covered with a protective tubing element 134. In some examples, protective tubing element 134 is also connected to active lead can 111. The protective tubing element 134 is configured to resist impact loads without shearing collapse, without high plastic deformation and skin penetration, while keeping the flexibility of the lead 130 comparable to the flexibility of the lead 130 without the protective tubing element 134. Furthermore, the total outer diameter is kept compatible with the implantable dimension, e.g., only at a few millimeters and at low wall thicknesses. Additionally, high axial stiffness is provided. Protective tubing element 134 includes a flexible outer tubing element and a structurally flexible reinforcement (not shown). In some examples, protective tubing element 134 also may include a flexible inner tuning element (not shown).

The protective tubing element 134 protects the proximal section of lead 130 manufactured based on thin film technology. In particular, the proximal part of the lead 134 is well protected, e.g., in the region of the cranium 128 and outside of the cranium 128 and the skull, from mechanical forces and stress. In some examples, the protective tubing element 134 covers a proximal part of the lead 130, while a distal portion of the lead 130 with the electrodes 132, is not covered by the protective tubing element 134. As discussed below with respect to FIG. 4, a plurality of conductors may extend from lead can 111 to distal electrodes 132 inside lead 130. In some examples, the length of protective tubing element 134 may be based on intended implant location. For example, protective tubing element 134 may be longer for procedures wherein lead 130 bends in order to place electrodes 132 in the desired location.

Protective tubing element 134 is flexible. In some examples, lead 130 is flexible. The flexibility of protective tubing element 134 allows it to bend along with lead 130 during implantation. The flexibility of protective tubing element 134 may also aid the protective tubing element in withstanding mechanical force without damage.

In some examples, the most distal part 136 of the protective tubing element 134 has a decreasing diameter. A decreasing diameter may be helpful during implantation of lead 130 and protective tubing element 134. For example, the decreasing diameter may define a taper in the diameter that provides a smoother introduction of the protective tube element 134 and lead 130 into tissue.

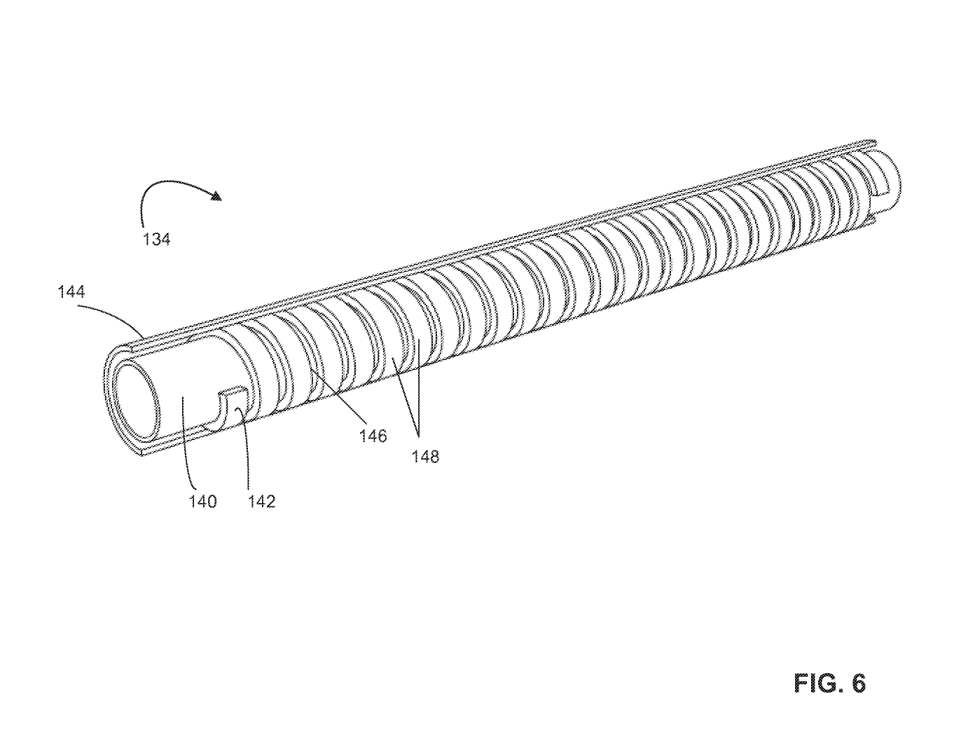

In some examples, protective lead element 134 is placed within burr hole 138 in cranium 128. For example, protective lead element 134 may positioned within burr hole 138 using a manner similar to catheter or other lead introducing element sheath. A clinician may use protective tubing element 134 as an introducer to guide the placement of lead 130 and electrodes 132 at a target location. Once protective lead element 134 has been placed within the burr hole, lead 130 may be fed through protective tubing element 134 until electrodes 132 reach the target location. In some examples, protective tubing element 134 may extend from outside burr hole 138 to an area proximate the target location for electrodes 132. In this manner, electrodes 132 are not covered by protective tubing element 134 once in place. In this way, protective tubing element 134 may protect lead 130 from the mechanical forces at burr hole 138. In other examples, protective tubing element 134 may cover the majority of lead 130, and travel along with lead 130 to a distal location, such as a target location for electrode 132. In some examples, protective tubing element 134 may be connected to lead 130 or to ALC 111. For example, one or more of the layers of protective tubing element 134 (as shown in FIG. 6, below) may be attached to ALC 111 along with lead 130. By traveling through tissue along with lead 130, protective tubing element 134 may protect lead 130 from mechanical forces not only at burr hole 138, but at bends in lead 130, for example.

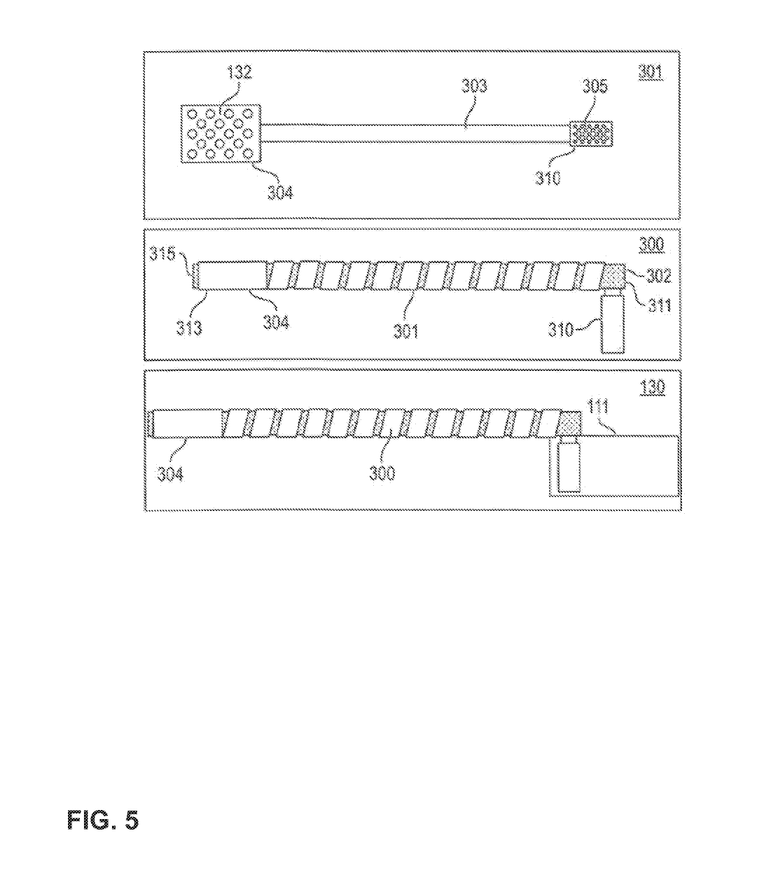

FIG. 5 further illustrates an example architecture for a DBS lead 130 that includes a proximal portion (shown in FIG. 2 as extension wire 120) probe 300 and an active lead can 111 comprising electronic means. The electronic means is provided to address electrodes 132 on the distal end 304 of the thin film 301, which is arranged at the distal end 313 and next to the distal tip 315 of the DBS probe 300. The probe 300 includes a carrier 302 for a thin film 301, said carrier 302 providing the mechanical configuration of the probe 300 and the thin film 301. The thin film 301 may include at least one electrically conductive layer, preferably made of a biocompatible material. The thin film 301 is assembled to the carrier 302 and further processed to constitute the probe 300. The thin film 301 for a lead is preferably formed by a thin film product having a distal end 304, a cable 303 with metal tracks and a proximal end 310. The proximal end 310 of the thin film 301 arranged at the proximal end 311 of the probe 300 is electrically connected to the active lead can 111. The active lead can 111 includes the switch matrix of the DBS steering electronics and may further include other electronics such as a pulse generator. The distal end 304 includes the electrodes 132 for the brain stimulation. The proximal end 310 includes the interconnect contacts 305 for each metal line in the cable 303. The cable 303 includes metal lines or tracks (not shown) to connect each distal electrodes 132 to a designated proximal contact 305.

The active lead can 111 may include an implantable pulse generator for delivery of neurostimulation via electrodes of lead 130, and/or one or more sensors configured to sense electrical fields within the brain of the patient 10, such as electrical fields representing a patient's brain activity and/or electrical fields created by delivery of DBS therapy. Alternatively, one or more of these functions may be provided by electronics in controller 110. In any event in various examples, either the same set of electrodes or different sets of electrodes may be used for sensing as those used for DBS therapy.

Although not shown in FIG. 5, a protective tubing element may be used in conjunction with active lead can 111 and lead 130. The protective tubing element may protect, thin film 301, for example, from forces during implantation. In some examples, the protective tubing element may extend from active can 111 toward distal electrodes 132. In some examples, an inner tube of the protective tubing element may provide insulation and prevent electrical contact between electrodes 132 and a structurally flexible reinforcement which may include metal.

FIG. 6 shows a perspective view of a protective tubing element 134. The protective tubing element 134 is a protective tubing element 134 for the lead 130 for neurostimulation and/or neurorecording as shown in FIGS. 2 and 4. The outer diameter of protective tubing element 134 may be between approximately 2.0 to 2.4 mm. In some examples, the outer diameter of protective tubing element 134 may up to approximately 5 mm.

The protective tubing element 134 includes an inner flexible tube 140 and an outer flexible tube 144. The inner flexible tube 140 and the outer flexible tube 144 may be made of a flexible polymer. The flexible polymer may be at lease partially an elastomer, a rubber, a thermoplastic urethane, a polyurethane or a polycarbonate-urethane, for example. In some examples, axial strain limit of the polymer is higher than 75%. The thickness of the wall of each of inner flexible tube 140 and outer flexible tube 144 may be between approximately 0.01 mm to approximately 0.5 mm. In some examples, the thickness may be between 0.05 mm to approximately 0.15 mm.

Structurally flexible reinforcement 142 may be arranged between and sandwiched by the inner flexible tube 140 and the outer flexible tube 144. The structurally flexible reinforcement 142 as shown in FIG. 5 is a coil. In some examples, the coil is a helically wound coil. The structurally flexible reinforcement 140 may be freely sandwiched between the least one inner flexible tube 140 and the at least one outer flexible tube 144, such that structurally flexible reinforcement 140 is not fixed to inner flexible tube 140 and outer flexible tube 144. As used in the present disclosure, freely sandwiched indicates there are no structural connections between structurally flexible reinforcement 142 and either inner flexible tube 140 or outer flexible tube 144. A lack of connections points between the structurally flexible reinforcement 142 and inner flexible tube 140 or outer flexible tube 144 allows all, or at least some portions, of structurally flexible reinforcement 142 to move, at least axially to some degree, with respect to either inner flexible tube 140 or outer flexible tube 144.

The structurally flexible reinforcement 326 may be at least partially made of metal. The metal is according to the shown example MP35N. In some examples, a metal alloy may be used, e.g. titanium.

The cross-section of the structurally flexible reinforcement 142, i.e., the cross-section of the winding of the coil of the structurally flexible reinforcement 142, may be substantially rectangular or rectangular, elliptical, circular, rhombic, trapezoidal, for example. The cross-sectional shape of the structurally flexible reinforcement 142 may be selected based on desired characteristics of the structurally flexible reinforcement 142. For example, a rectangular cross-sectioned coil may provide greater stability against sheering under force than a circular cross-sectioned coil. The structurally flexible reinforcement 142 protects the lead from external forces while maintaining flexibility to be implanted in patient.

In some examples, not shown, the structurally flexible reinforcement 142 may take other essentially tube-like structures. For example, the structurally flexible reinforcement 142 may be constructed of a plurality of interlocking rings, or a braided mesh tube.

The structurally flexible reinforcement 142 provides interspaces 146 between its windings 148. The interspaces 146 may be formed by the pitch of the coil forming the structurally flexible reinforcement 142. In some examples, the interspaces 146 may be not filled, e.g., there may be, for example, no polymer matrix or glue matrix. In some examples, the interspaces can be filled with compressible material, e.g., air or a compressible gas or fluid. In general, any low stiffness compressible material can be used.

The pitch of the coil may be, for example, in the range of the width or between a range of 0.5 to 1.5 times the width of wire forming the coil. For example, the pitch of the coil may be in the range of the width of the windings of the coil at around 1.2 times of the width of the wire forming the coil.

In some examples, instead of being entirely free relative to the inner tube 140 and outer tube 144, the structurally flexible reinforcement 142 is partially attached to the inner and/or outer tube 140, 144 by means of gluing, for example. However, in examples including a connection between structurally flexible reinforcement 142 and the inner and outer tube 140, 144, structurally flexible reinforcement 142 may be still able to perform relative movements with respect to inner tube 140 and outer tube 144. In order to achieve movement relative to the inner and outer tubes 140, 144, connections (not shown) between structurally flexible reinforcement 142 and inner tube and outer tubes 140, 144 may be spaced along the length of protective tubing element 134. In some examples, the thickness of structurally flexible reinforcement 142 is between approximately 0.25 mm and 1.25 mm. In some examples, the thickness of structurally flexible reinforcement 142 is between 0.5 mm to approx. 1 mm.

In some examples, inner tube 140 may include a lubricious substrate layer. The lubrication may be between the inner tube 140 and a lead (not shown). The lubricious substrate layer is comprised of HDPE, PTFE and/or PEEK. The lubricous substrate layer reduces surface friction between a lead and the protective tubing element 134.

Although not shown in FIG. 6, protective tubing element 134 may include an attachment mechanism to enable attachment to an introducer system or to ALC 111. For example, the proximal end of protective tubing element 134 may include a mechanism by which to attach to a dilator while being introduced in the body of patient 10. In other examples, 134 may mate with ALC 111 around lead 130.

In some examples, the structurally flexible reinforcement 142 is laser cut from a metal tube, and then inner tube 140 is extruded within structurally flexible reinforcement 142. Connections between inner tube 140 and structurally flexible reinforcement 142 are created after inner tube 140 is in place. In other examples, inner tube 140 is threaded in to structurally flexible reinforcement 142 and then bonded at spaced intervals along the length of structurally flexible reinforcement 142. In addition, outer tube 144 may be extruded over structurally flexible reinforcement 142, or threaded over structurally flexible reinforcement 142. Connections between structurally flexible reinforcement 142 and outer tube 144 are created at spaced intervals in order to allow structurally flexible reinforcement 142 to move freely with respect to both inner tube 140 and outer tube 144.

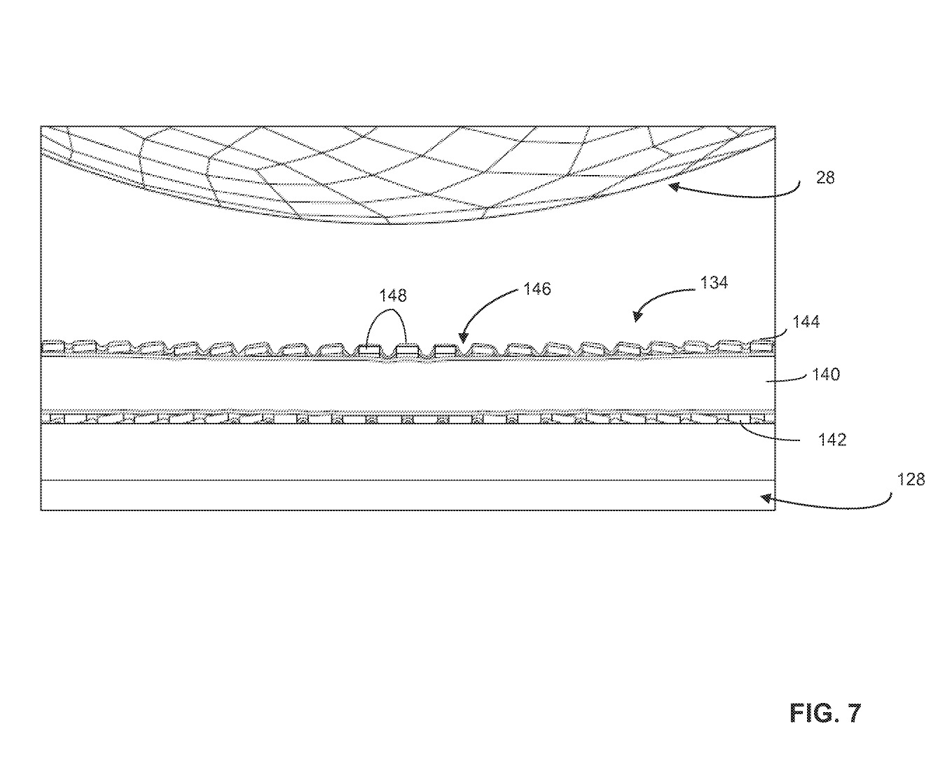

FIG. 7 shows a cross-sectional side view of the protective tubing element 134 in the moment after an impact as protective tubing element is inserted between cranium 128 of patient 10 and brain 28 of patient 10. Interspaces 146 of the coil of structurally flexible reinforcement 142 provide enough space for outer tube 144, for example, to partially deform in the direction of the structurally flexible reinforcement 142, in response to impact or mechanical force or bending. As shown in FIG. 7, the forces on protective tubing element 134 upon impact cause outer tube 144 to be pressed against structurally flexible reinforcement 142. In embodiments of protective tubing element 134 including an inner tube 140, outer tube 144 may be pressed through interspaces 146 until coming in contact with inner tube 144. The lack of connections points between windings 148 and outer tube 144 allows outer tube 144 to deform within interspaces 146 without also deforming structurally flexible reinforcement 142.

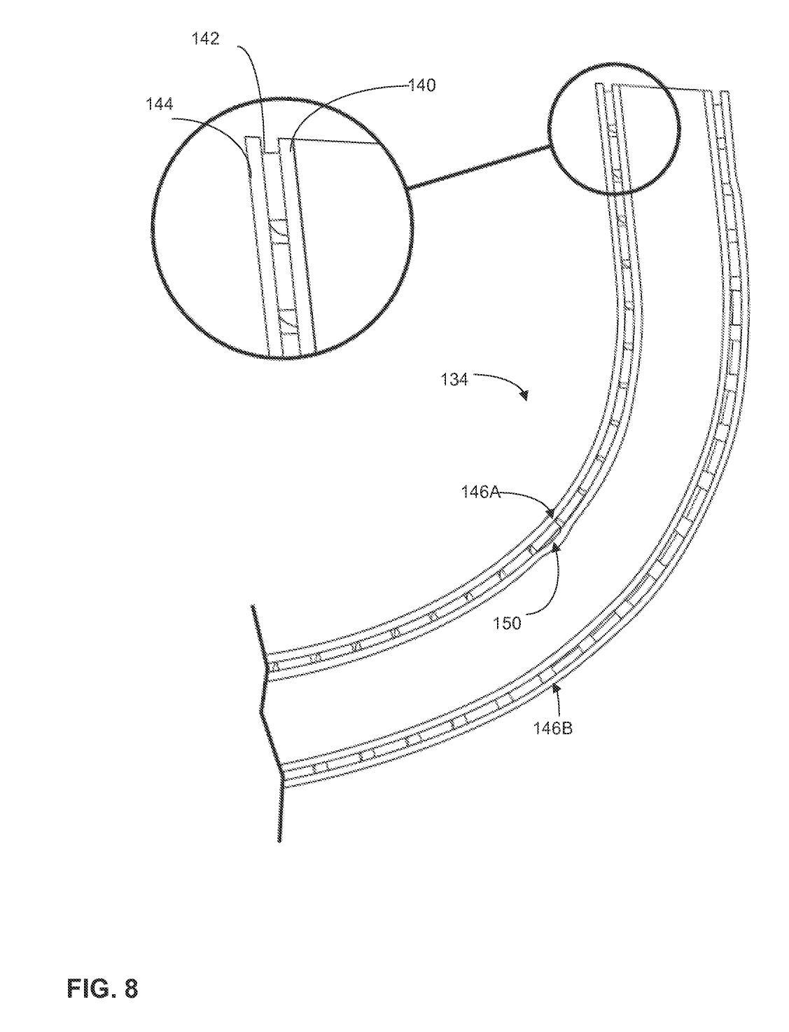

FIG. 8 shows a cross-sectional side view of the protective tubing element 134 in the moment of bending. Protective tubing element 134 includes outer tube 144, structurally flexible reinforcement 142, and, optionally, inner tube 140. Structurally flexible reinforcement 142 is freely sandwiched between outer tubing 144 and inner tubing 140. In some examples, not shown, structurally flexible reinforcement 142 is within outer tubing 144, without inner tubing 140 being present. When protective tubing element 134 bends, the interspaces 146 change. For example, as shown in FIG. 8, interspace 146A, which is on the interior side of the bend, becomes smaller, while interspace 146B, which is on the exterior side of the bend, becomes larger. Because outer tube 144 may move freely with respect to structurally flexible reinforcement 142, portions of outer tube 144 do not get caught in interspace 146A. Instead, outer tube 144 glides over the windings of structurally flexible reinforcement 142. Further, the lack of connection between structurally flexible reinforcement 142 and inner tube 140 allows for gap 150 to form. Both gap 150 and the lack or pinching of outer tube 144 are a result of outer tube 144, inner tube 140 and structurally flexible reinforcement 142 moving freely with respect to one another. The free movement prevents damage to protective tubing element 134 including, for examples, kinking or bulging. In some examples, lubrication may be present between the layers of protective tubing element 134 to aid in the free movement of the inner tube 140, the structurally flexible reinforcement 142, and outer tube 144.

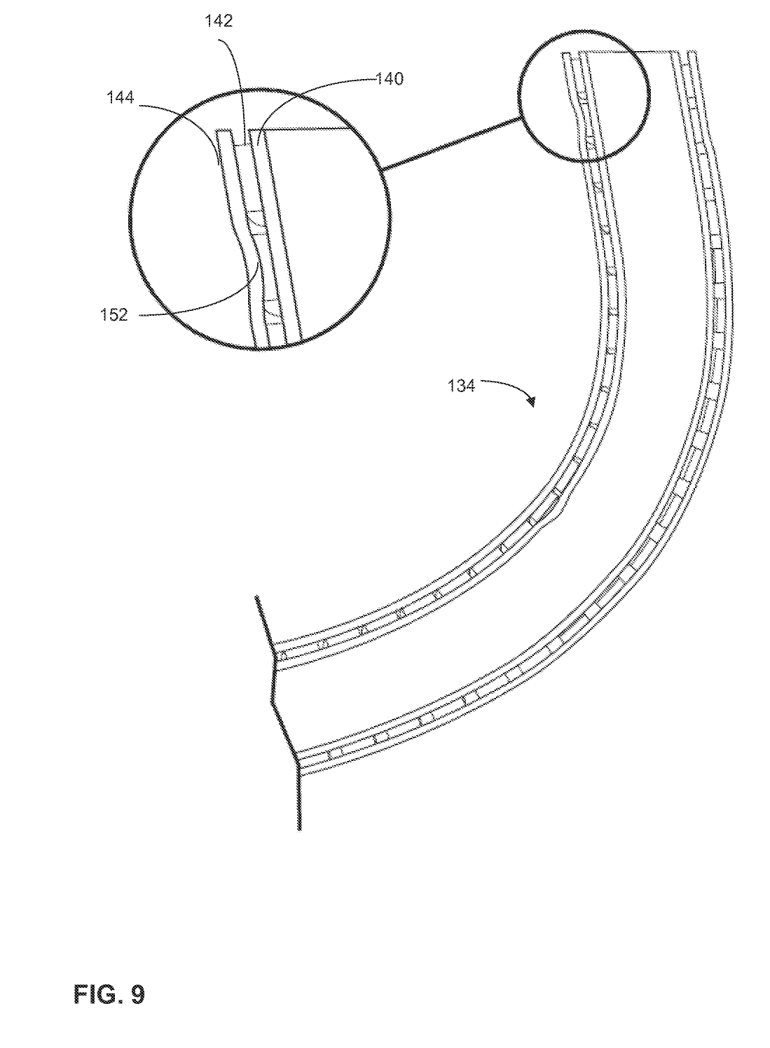

FIG. 9 shows a cross-sectional side view of the protective tubing element 134 in the moment of bending. Protective tubing element 134 includes outer tube 144, structurally flexible reinforcement 142, and, optionally, inner tube 140. Structurally flexible reinforcement 142 is freely sandwiched between outer tubing 144 and inner tubing 140. As shown in FIG. 8, there is at least one connection 152 between outer tubing 144 and a winding of structurally flexible reinforcement 142. The connection may be made using glue or thermal bonding, mechanical fastening or welding, for example. Connection 152 may be a single connection between outer tubing 144 and structurally flexible reinforcement 142 at the distal end of protective tubing element 134. In other examples, protective tubing element 134 may include a plurality of connections 152 between structurally flexible reinforcement 142 and outer tubing 144, or between structurally flexible reinforcement 142 and inner tubing 140, or both. For example, a plurality of connections 152 may be dispersed evenly along the length of protective tubing element 134. In some examples, the spacing may be determined based on the radius of bending the protective tubing element is predicted to encounter. In such examples, the space between connections 152 may be large enough to ensure no more than one connection is present within the bent portion of protective tubing element 134. In this manner, outer tube 144 and structurally flexible reinforcement 142 may still move relatively freely with respect to one another, e.g., between connections. In some examples, the spacing between connections 152 may be random. In some examples, two connections 152 may be present in protective tubing element 134. A first connection at the proximal end of the protective tubing element 134 and a second connection at the distal end of protective tubing element 134. The two connections allow outer tube 144 and structurally flexible reinforcement 142 to move freely with respect to one another while still being connected.

Examples of this disclosure may be applied to, for example, catheters with intelligent tips, leads for DBS, cochlear implants, alike in ear hearing aids, pacemakers, implantable cardiac defibrillators, spinal cord stimulators, gastric stimulators, pelvic floor stimulators, and other implantable systems including stimulation and/or sensing functions.

Various examples have been described. These and other examples are within the scope of the following claims.

What is claimed is:

1. A medical lead comprising
medical lead; and
a protective tubing element comprising:
an outer flexible tube; and
a structurally flexible reinforcement at least partially covered by the outer flexible tube,
wherein the structurally flexible reinforcement and the outer flexible tube are arranged such that at least some portions of the structurally flexible reinforcement and the outer flexible tube are moveable relative to each other; and wherein the protective tubing element is configured to cover a proximal portion of the medical lead.

2. The medical lead of claim 1, wherein the structurally flexible reinforcement includes a coil.

3. The medical lead of claim 1, further comprising an inner flexible tube on a side of the structurally flexible reinforcement opposite the outer flexible tube, wherein the structurally flexible reinforcement and the inner flexible tube are arranged such that at least some portions of the structurally flexible reinforcement and the inner flexible tube are moveable relative to each other.

4. The medical lead of claim 3, wherein the structurally flexible reinforcement is freely sandwiched between the inner flexible tube and the outer flexible tube.

5. The medical lead of claim 3, further comprising a plurality of connection points along a length of the structurally flexible reinforcement, the connection points providing structural connections between portions of the structurally flexible reinforcement and at least one of the inner flexible tube and the outer flexible tube.

6. The medical lead of claim 1, wherein the structurally flexible reinforcement is coupled to the outer flexible tube at least one of: at a proximal end of the outer flexible tube or at a distal end of the outer flexible tube.

7. The medical lead of claim 1, wherein the outer flexible tube is at least partially made of a flexible polymer.

8. The medical lead of claim 7, wherein the flexible polymer comprises at least one of: an elastomer, a rubber, a thermoplastic urethane, a polyurethane or a polycarbonate-urethane.

9. The medical lead of claim 8, wherein an axial strain limit of the polymer is higher than 50%.

10. The medical lead of claim 1, wherein the structurally flexible reinforcement includes a braided mesh.

11. The medical lead of claim 1, wherein the structurally flexible reinforcement is at least partially made of metal or a metal alloy.

12. The medical lead of claims 11, wherein the metal is titanium or wherein the metal alloy is a nickel alloy.

13. The medical lead of claim 1, wherein the cross-section of the least one structurally flexible reinforcement is substantially rectangular, elliptical, circular, rhombic, or trapezoidal.

14. The medical lead of claim 1, further configured to connect to an active lead can.

15. A system, comprising:
a medical lead comprising a lead body having a proximal end and a distal end;
a plurality of electrodes at the distal end of the lead body;
a plurality of conductors extending from the electrodes to the proximal end of the lead body;
a controller configured to deliver electrical stimulation current via the plurality of electrodes of the medical lead; and
a protective tubing element comprising:
an outer flexible tube; and
a structurally flexible reinforcement at least partially covered by the outer flexible tube, wherein the structurally flexible reinforcement and the outer flexible tube are arranged such that the structurally flexible reinforcement and the outer flexible tube allow movements relative to each other, and wherein the protective tubing element is configured to cover a proximal portion of the medical lead.

16. The system of claim 15, wherein the controller includes an implantable pulse generator for delivery of neurostimulation via the plurality of electrodes.

17. The system of claim 16, further comprising an active lead can carried by the medical lead, and wherein the active lead can comprises the controller.

18. The system of claim 16, further comprising an implantable medical device operatively coupled to the medical lead, and wherein the implantable medical device comprises the controller.

19. The system of claim 16, wherein the controller includes a sensor configured to sense electrical fields within a brain of the patient via the plurality of electrodes.

20. The system of claim 15, further comprising an inner flexible tube on a side of the structurally flexible reinforcement opposite the outer flexible tube, wherein the structurally flexible reinforcement and the inner flexible tube are arranged such that at least some portions of the structurally flexible reinforcement and the inner flexible tube are moveable relative to each other.

21. The system of claim 20, wherein the structurally flexible reinforcement is freely sandwiched between the inner flexible tube and the outer flexible tube.

22. The system of claim 15, wherein the structurally flexible reinforcement is coupled to the outer flexible tube at least one of: at a proximal end of the outer flexible tube or at a distal end of the outer flexible tube.

23. The system of claim 15, further comprising a plurality of connection points along the length of the structurally flexible reinforcement, the connection points providing structural connections between portions of the structurally flexible reinforcement and at least one of the inner flexible tube and the outer flexible tube.

24. A system comprising:
a medical lead; and
a protective tubing element comprising:
an outer flexible tube;
an inner flexible tube; and
a structurally flexible reinforcement sandwiched between the outer flexible tube and the inner flexible tube, wherein the structurally flexible reinforcement and the outer flexible tube are arranged such that the structurally flexible reinforcement and the outer flexible tube allow movements relative to each other, and wherein the protective tubing element is configured to cover a proximal portion of the medical lead.

25. The system of claim 22 further comprising an implantable medical device comprising a pulse generator.

26. The system of claim 23, wherein the pulse generator comprises an active lead can.
